(19) United States
(12) Patent Application Publication
(10) Pub. No.: US 2018/0116858 A1
(43) Pub. Date: May 3, 2018

(54) Restraining Garment Assembly

(71) Applicant: Merry Martin, Norwood, NC (US)

(72) Inventor: Merry Martin, Norwood, NC (US)

(21) Appl. No.: 15/341,442

(22) Filed: Nov. 2, 2016

(51) Int. Cl. A61F 5/37; A41D 1/06

(52) U.S. Cl. CPC A61F 5/3715; A41D 1/06

(57) ABSTRACT

A restraining garment assembly for restraining a paralyzed user's legs includes a pair of pants that may be worn. The pair of pants include a first leg and a second leg. A pair of first straps is provided and each of the first straps is coupled to the first leg. A pair of second straps is provided and each of the second straps is coupled to the second leg. Each of the second straps is matable with the first straps. Thus, each of the first straps and the second straps inhibit legs of a paralyzed user from being moved apart from each other thereby inhibiting the paralyzed user's legs from being injured.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art including information disclosed under 37 CFR 1.97 and 1.98.

The disclosure and prior art relates to garment devices and more particularly pertains to a new garment device for restraining a paralyzed user's legs.

### BRIEF SUMMARY OF THE INVENTION

An embodiment of the disclosure meets the needs presented above by generally comprising a pair of pants that may be worn. The pair of pants include a first leg and a second leg. A pair of first straps is provided and each of the first straps is coupled to the first leg. A pair of second straps is provided and each of the second straps is coupled to the second leg. Each of the second straps is matable with the first straps. Thus, each of the first straps and the second straps inhibit legs of a paralyzed user from being moved apart from each other thereby inhibiting the paralyzed user's legs from being injured.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a restraining garment assembly according to an embodiment of the disclosure.

FIG. 2 is a front view of an embodiment of the disclosure.

FIG. 3 is a back view of an embodiment of the disclosure.

FIG. 4 is a perspective in-use view of an embodiment of the disclosure.

### DETAILED DESCRIPTION OF THE INVENTION

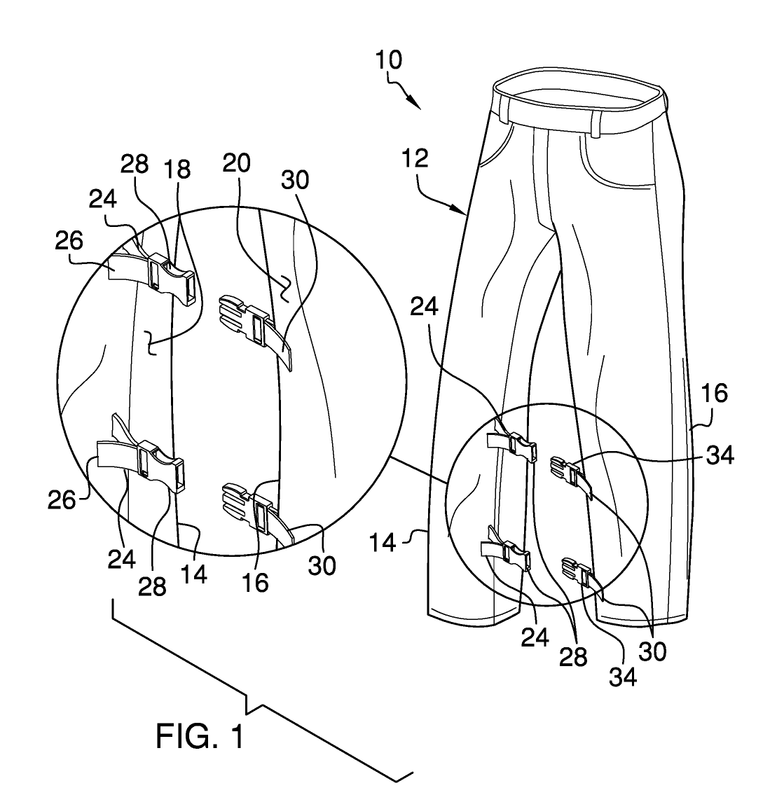

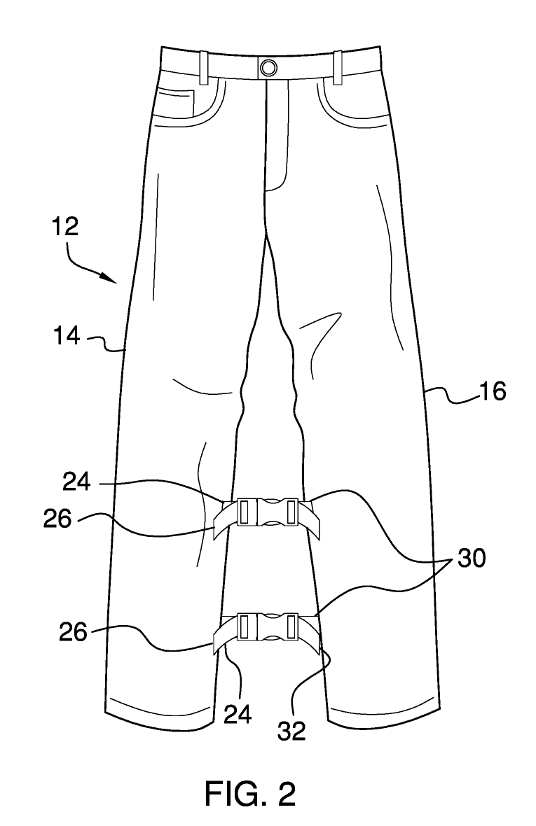

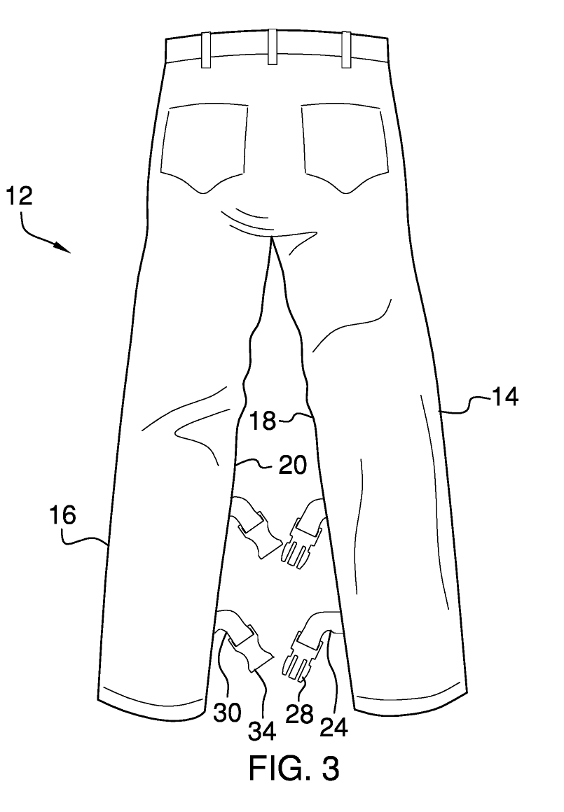

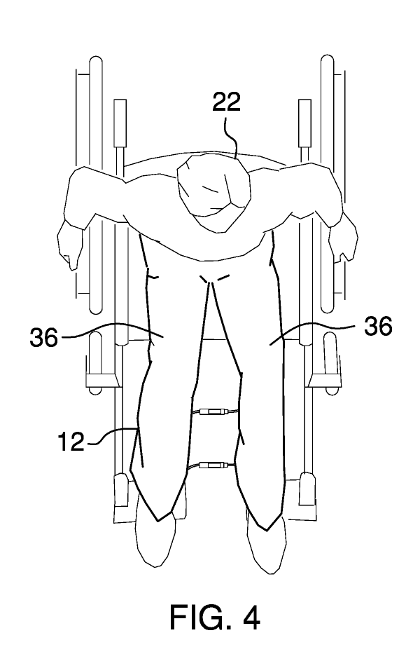

With reference now to the drawings, and in particular to FIGS. 1 through 4 thereof, a new garment device embodying the principles and concepts of an embodiment of the disclosure and generally designated by the reference numeral 10 will be described.

As best illustrated in FIGS. 1 through 4, the restraining garment assembly 10 generally comprises a pair of pants 12 that may be worn. The pair of pants 12 includes a first leg 14 and a second leg 16. The first leg 14 has a first surface 18 and the second leg 16 has a first surface 20. The first surface 18 corresponding to the first leg 14 faces the first surface 20 corresponding to the second leg 16. The pair of pants 12 may be jeans, slacks or other wearable pants 12. Moreover, the pair of pants 12 is worn by a paralyzed user 22.

A pair of first straps 24 is provided. Each of the first straps 24 is coupled to the first leg 14 and each of the first straps 24 may be manipulated. Each of the first straps 24 is positioned on the first surface 18 corresponding to the first leg 14. Moreover, each of the first straps 24 has a distal end 26 with respect to the first leg 14. A pair of first fasteners 28 is provided. Each of the first fasteners 28 is slidably coupled to the distal end 26 of an associated one of the first straps 24.

A pair of second straps 30 is provided. Each of the second straps 30 is coupled to the second leg 16 and each of the second straps 30 may be manipulated. Each of the second straps 30 is positioned on the first surface 20 corresponding to the second leg 16. Moreover, each of the second straps 30 has a distal end 32 with respect to the second leg 16. Each of the second straps 30 is aligned with an associated one of the first straps 24.

A pair of second fasteners 34 is provided. Each of the second fasteners 34 is slidably coupled to the distal end 32 of an associated one of the second straps 30. Moreover, each of the second fasteners 34 is complementary with respect to the first fasteners 28. Thus, each of the first straps 24 and the second straps 30 inhibit legs 36 of a paralyzed user 22 from being moved apart from each other. Thus, the first straps 24 and the second straps 30 inhibit the paralyzed user's legs 36 from being injured. Each of the first fasteners 28 and the second fasteners 34 may be belt buckles or the like.

In use, the pants 12 are worn by the paralyzed user 22. Each of the first straps 24 is coupled to an associated one of the first straps 24. Thus, each of the paralyzed user's legs 36 is inhibited from being moved apart from each other. The pants 12 facilitate the paralyzed user 22 to be transported to a wheelchair while restraining the paralyzed user's legs 36. Moreover, the pants 12 inhibit the paralyzed user's legs 36 from falling off of footrests on the wheelchair. Thus, the pants 12 inhibit the paralyzed user's legs 36 from being injured when the paralyzed user 22 is in the wheelchair. Additionally, the pants 12 inhibits the paralyzed user's legs 36 from moving due to involuntary muscle spasms.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A restraining garment assembly being configured to be worn by a paralyzed person thereby facilitating legs of the paralyzed person to be restrained, said assembly comprising:
a pair of pants being configured to be worn, said pair of pants including a first leg and a second leg;
a pair of first straps, each of said first straps being coupled to said first leg wherein each of said first straps is configured to be manipulated; and
a pair of second straps, each of said second straps being coupled to said second leg wherein each of said second straps is configured to be manipulated, each of said second straps being matable with said first straps wherein each of said first straps and said second straps are configured to inhibit legs of a paralyzed user from being moved apart from each other thereby inhibiting the paralyzed user's legs from being injured.

2. The assembly according to claim 1, wherein:
said first leg has a first surface; and
said second leg has a first surface, said first surface corresponding to said first leg facing said first surface corresponding to said second leg.

3. The assembly according to claim 2, further comprising each of said first straps is positioned on said first surface corresponding to said first leg, each of said first straps having a distal end with respect to said first leg.

4. The assembly according to claim 3, further comprising a pair of first fasteners, each of said first fasteners being coupled to said distal end of an associated one of said first straps.

5. The assembly according to claim 2, wherein each of said second straps is positioned on said first surface corresponding to said second leg, each of said second straps having a distal end with respect to said second leg.

6. The assembly according to claim 5, further comprising:
a pair of first fasteners; and
a pair of second fasteners, each of said second fasteners being coupled to said distal end of an associated one of said second straps, each of said second fasteners being complementary with respect to said first fasteners.

7. A restraining garment assembly being configured to be worn by a paralyzed person thereby facilitating legs of the paralyzed person to be restrained, said assembly comprising:
a pair of pants being configured to be worn, said pair of pants including a first leg and a second leg, said first leg having a first surface, said second leg having a first surface, said first surface corresponding to said first leg facing said first surface corresponding to said second leg;
a pair of first straps, each of said first straps being coupled to said first leg wherein each of said first straps is configured to be manipulated, each of said first straps being positioned on said first surface corresponding to said first leg, each of said first straps having a distal end with respect to said first leg;
a pair of first fasteners, each of said first fasteners being coupled to said distal end of an associated one of said first straps;
a pair of second straps, each of said second straps being coupled to said second leg wherein each of said second straps is configured to be manipulated, each of said second straps being positioned on said first surface corresponding to said second leg, each of said second straps having a distal end with respect to said second leg; and
a pair of second fasteners, each of said second fasteners being coupled to said distal end of an associated one of said second straps, each of said second fasteners being complementary with respect to said first fasteners wherein each of said first straps and said second straps are configured to inhibits legs of a paralyzed user from being moved apart from each other thereby inhibiting the paralyzed user's legs from being injured.
